(19) United States
(12) Patent Application Publication
(10) Pub. No.: US 2009/0209861 A1
(43) Pub. Date: Aug. 20, 2009

(54) Ultrasonic Doppler Blood Flow Meter

(75) Inventors: Morio Nishigaki, Kanagawa (JP); Hisashi Hagiwara, Kanagawa (JP); Takenori Fukumoto, Kanagawa (JP)

Correspondence Address: WENDEROTH, LIND & PONACK L.L.P., 1030 15th Street, N.W., Suite 400 East, Washington, DC, 20005-1503, US

(73) Assignee: PANASONIC CORPORATION, Osaka (JP)

(21) Appl. No.: 11/884,440

(22) PCT Filed: Feb. 16, 2006
(86) PCT No.: PCT/JP2006/302734

§ 371 Date: Oct. 21, 2008

(30) Foreign Application Priority Data
Feb. 17, 2005 (JP) 2005-040189

(51) Int. Cl. A61B 8/06

(52) U.S. Cl. 600/455

(57) ABSTRACT

An ultrasonic Doppler blood flow meter has a clutter component estimation unit 110 for estimating amplitude, initial phase, and phase velocity of the clutter component using an output from the phase detector 104 to reduce a transient phenomenon. Using the estimated data, initial values of the wall filter 105 are generated by the initial filter value generating unit 11 and then provided to the wall filter for the filtering process.

### BACKGROUND OF THE INVENTION

The present invention relates to an ultrasonic Doppler blood flow meter for use in the medical field for measuring a blood flow in the body by using Doppler effect and then displaying the measured blood flow data.

### SUMMARY OF THE INVENTION

There has been known an ultrasonic Doppler blood flow meter (color flow meter) from, such as, the Japanese Patent Publication No. 11-299786 A. The blood flow meter uses the Doppler effect of the ultrasonic wave and displays a distribution of blood flows with respective colors as it is superimposed on a two-dimensional monochrome tomographic image.

FIG. 6 shows a general construction of the ultrasonic Doppler blood flow meter, generally indicated at 10A. According to the illustrated ultrasonic flow meter 10A, a transmitter 1 transmits a series of ultrasonic pulses through a probe 2 into the body of the subject being tested. An echo from the body of the transmitted ultrasonic pulses is transformed into an electric signal by the probe 2, which in turn is transported into a receiver 3. The receiver 3 amplifies the received faint signal and then converts the amplified signal from analogue to digital form by an analogue to digital converter not shown. Subsequently, the digital signal is transmitted into a phase detector 4 where it is converted into a basebanded echo signal. The basebanded echo signal is transmitted into a wall filter 5. The wall filter 5, which typically includes FIR filter or IIR filter, eliminates unnecessary low frequency component of the signal reflected from tissues of the body, called “clutter.” The clutter-eliminated echo signal is transmitted from the wall filter 5 to a blood flow data arithmetic unit (flow rate calculation unit) 6 where it is used for the calculation of the blood flow rate. The calculated blood flow rate is transmitted into a digital scan converter 8 (hereinafter referred to as “DSC”) into which B-mode signal is also transmitted from an envelope detector 7. The DSC 8 combines the B-mode signal with the blood flow signal. The combined signal is then used for displaying a two-dimensional blood flow image on a monitor 9.

FIG. 7 shows details of the wall filter using a secondary IIR filter. In the drawing, x(m) denotes an input signal (input) for the wall filter, y(m) denotes an output signal (output) from the wall filter 5, 20₁-20₃ denote adders, 21ₐ₂-21ₐ₃ and 21_{b1-21}_{b3} are multipliers, 22₁-22₂ are memory elements (registers), and a(2)-a(3) and b(1)-b(3) are filter coefficients.

For the ultrasonic Doppler blood flow meter, it is preferable that the wall filter 5 has a sharp characteristic in order to generate highly accurate blood flow data. The sharp characteristic, however, results in greater transient response. Data including that transient response tends to prevent from obtaining accurate blood flow data. For this reason, a larger part of data is destructed unusedly in order to eliminate the affect of the transient response and thereby obtain accurate blood flow rate. This also reduces the amount of data to be transmitted to the flow rate calculation unit and also reduces the number of averaging procedures, which results in a deterioration of signal/noise ratio.

An increase of the number of data sampling operations for the compensation of data destruction results in another increase in the repetition number of transmitting and receiving of ultrasonic wave, which in turn decreases the frame rate.

Further, a cutoff frequency of the filter in the conventional ultrasonic Doppler blood flow meter is so designed that the filter well cuts off clutters with lower frequencies delivered from the slow-moving portions of the body, such as low-beating heart. However, the filter fails to cut off clutters from the rapid-moving portions the body, such as high-beating heart, because the clutters have frequencies higher than the cutoff frequency of the filter, which results in a disadvantageous error in the calculation of the blood flow rate. To raise the cutoff frequency above the uppermost frequency of the clutters would result in that the blood flow is unable to be displayed because the blood flow frequency in the slow-moving condition becomes lower than the cutoff frequency.

### SUMMARY OF THE INVENTION

The present invention is to solve such problems and provide an ultrasonic Doppler blood flow meter which uses a wall filter with a sharp characteristic and capable of obtaining a filtered output with a lot less transient response and thereby displaying the blood flow in an excellent manner.

Also, the present invention is to overcome the conventional drawbacks and provide an ultrasonic Doppler blood flow meter capable of eliminating clutters irrespective of the variation in frequency thereof from the body portions moving rapidly, performing a filtering process for the extraction of the blood signal, and thereby displaying the blood flow in an excellent manner.

To attain this, the ultrasonic Doppler blood flow meter according to the present invention uses plural echo signals responsive to the plural ultrasonic waves transmitted in the subject body to approximate the amplitude, velocity, and phase of the clutter component in the form of sinusoidal wave with a single frequency. The approximated clutter component is transmitted constantly to the wall filter. Data to be accumulated in the resisters of the wall filter is determined at timings for obtaining the echo signals, which is then used for the setting of the wall filer. This prevents a discontinuity in the signals and reduces unnecessary component due to the transient response, obtaining reliable blood flow data. For this purpose, the ultrasonic Doppler blood flow meter of the present invention, which comprises

a transmitter for generating electric pulses;

a probe for receiving the electric pulses and transforming the electric pulses, the probe transmitting the transformed electric pulses into a body or subject to be tested and then receiving a signal including an echo signal responsive to the electric pulses from the body;

a phase detector for phase-detecting the echo signal from the signal received by the probe;

a wall filter for eliminating a low frequency clutter component from the echo signal detected by the phase detector;

a blood flow data calculating unit for calculating blood flow data from an output in which the low frequency clutter component has been eliminated;

a digital scan converter for transforming the blood flow data from the blood flow data calculating unit into another form of signal; and

a monitor for using the another form of signal to display blood flow data;

is characterized in that

the ultrasonic Doppler blood flow meter further comprises

a clutter component estimation unit for approximating the clutter component by a sinusoidal wave by using at least two signals from the phase detector; and

an initial filter value generating unit for generating initial values of the wall filter by using the sinusoidal wave approximated by the clutter component estimation unit.

In another aspect of the present invention, the ultrasonic Doppler blood flow meter is characterized in that it has a cutoff frequency setting unit for setting a cutoff frequency of the wall filter from the estimated clutter component, which is capable of using the velocity of the clutter component approximated by the single frequency, sinusoidal wave to set a cutoff frequency of the wall filter so that the wall filter eliminates the clutter component, ensuring that the clutter component is always eliminated and only the blood flow data is extracted.

In another aspect of the present invention, the ultrasonic Doppler blood flow meter is characterized in that it has a cutoff frequency setting unit for setting a cutoff frequency of the wall filter from the estimated clutter component, which is capable of determining whether the approximated clutter signal has bee derived from the clutter or the blood blow from the amplitude of the single-frequency, approximated clutter component, ensuring that the clutter component is always eliminated and only the blood flow data is extracted.

In another aspect of the present invention, the ultrasonic Doppler blood flow meter is characterized in that it has a clutter differentiating unit whether an amplitude of the estimated clutter component indicates the clutter component or the blood flow data from the amplitude information in a region adjacent to another region where it is under scanning or the amplitude information of the clutter component obtained by a previous scanning of the same region, which is capable of determining whether the approximated clutter signal has been derived from the clutter, ensuring that the clutter component is always eliminated and only the blood flow data is extracted.

According to the ultrasonic Doppler blood flow meter so constructed according to the present invention, a filter is provided which prevents the transient response caused by the clutters with large amplitude, allowing to obtain an accurate blood flow data.

### BRIEF DESCRIPTION OF THE DRAWINGS

FIG. 1 is a block diagram showing a construction of an ultrasonic Doppler blood flow meter according to the first embodiment of the present invention;

FIG. 2 is a diagram showing a part of the wall filter for preventing a transient response of the ultrasonic Doppler blood flow meter shown in FIG. 1;

FIG. 3 is a block diagram showing a construction of an ultrasonic Doppler blood flow meter according to the second embodiment of the present invention;

FIG. 4 is a block diagram showing a construction of an ultrasonic Doppler blood flow meter according to the third embodiment of the present invention;

FIG. 5 is a block diagram showing a construction of an ultrasonic Doppler blood flow meter according to the fourth embodiment of the present invention;

FIG. 6 is a block diagram showing a construction of the conventional ultrasonic Doppler blood flow meter; and

FIG. 7 is a block diagram showing a construction of the ultrasonic Doppler blood flow meter shown in FIG. 6.

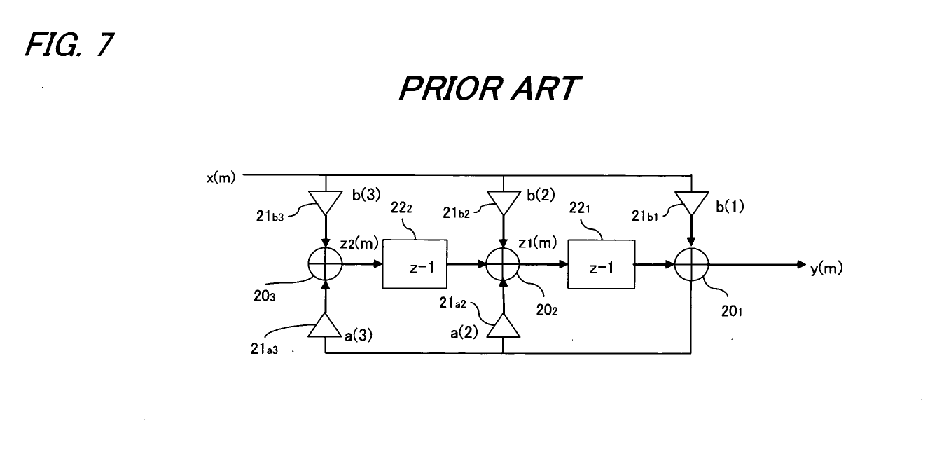

### DESCRIPTION OF THE REFERENCE NUMERALS

1, 101: transmitter
2, 102: probe
3, 103: receiver
4, 104: phase detector
5, 105A, 105B: wall filter
6, 106: blood flow data calculation unit
7, 107: envelope detector
8, 108: DSC
9, 109: monitor
110: clutter component estimation unit
111: initial filter coefficient generating unit
112: cutoff frequency setting unit
113: filter coefficient setting unit
115: clutter differentiating unit
116: clutter information accumulating unit

### DETAILED DESCRIPTION OF THE PREFERRED EMBODIMENTS

With reference to the accompanying drawings, an embodiment of the present invention will be described below.

### First Embodiment

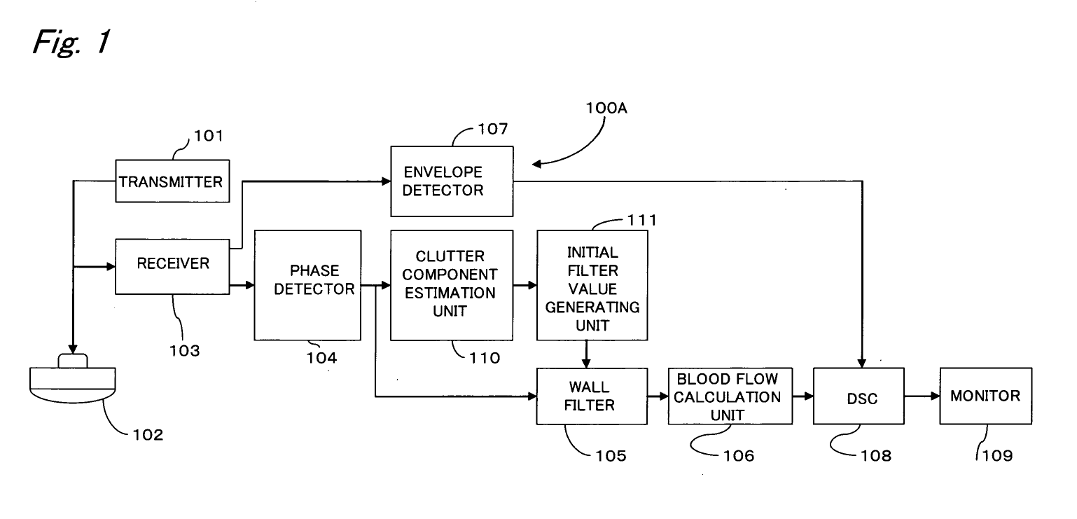

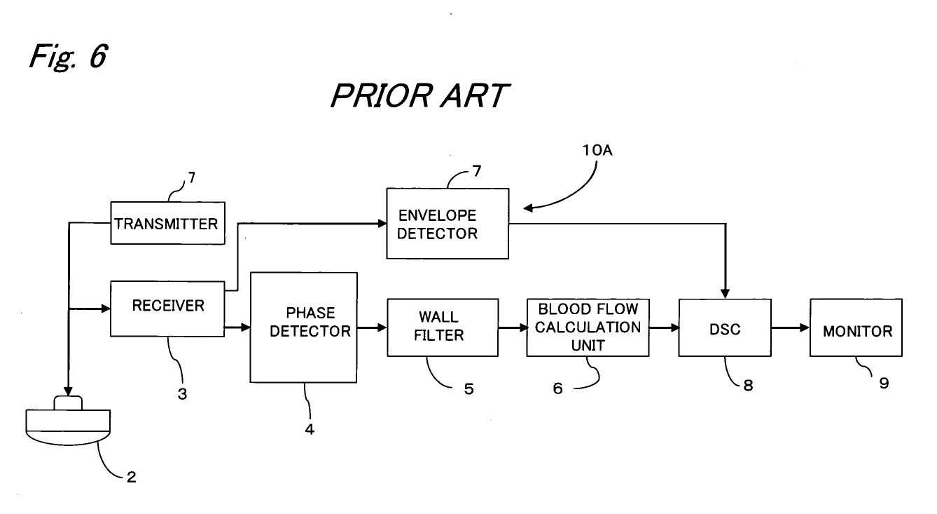

FIG. 1 is a block diagram showing a general construction of an ultrasonic Doppler blood flow meter according to the first embodiment of the present invention. The illustrated ultrasonic Doppler blood flow meter, generally indicated at 100A, differs from the conventional blood flow meter described with reference to FIG. 6 in that the blood flow meter 100A has a clutter component estimation unit 110 and an initial filter value generating unit 111 both added thereto.

In operation of the blood flow meter 100A so constructed, the transmitter 101 transmits ultrasonic pulses through the probe 102 into the body of the subject to be tested. An echo from the body responsive to the transmitted ultrasonic pulses is transformed into an electric signal by the probe 102, which is in turn transmitted into the receiver 103. The receiver 103 amplifies the received faint signal and then converts the signal from analogue to digital form by an analogue to digital converter not shown. Subsequently, the digital signal is transmitted into the phase detector 104 where it is converted into a basebanded echo signal. The basebanded echo signal is transmitted into the wall filter 105 and the clutter component estimation unit 110. Using the output from the phase detector 104, the clutter component estimation unit 110 estimates the clutter component. The initial filter value generating unit 111 uses the estimated clutter component to generate initial filter value which are delivered to the wall filter 105. The wall filter 105, which typically includes FIR filter or IIR filter, uses the initial filter values from the initial filter value generating unit 111 to eliminate the unnecessary low frequency component of the signal reflected from tissues of the body, called “clutter.” The echo signal from which the clutter has been eliminated is transmitted to the blood flow data arithmetic unit (flow rate calculation unit) 106 where the flow rate of the blood flow is calculated. The calculated blood flow rate is transmitted into the digital scan converter 108 (hereinafter referred to as “DSC”) into which B-mode signal is also transmitted from an envelope detector 107. The DSC 8 combines the B-mode signal with the blood flow signal. Using the combined signal, the monitor 109 displays a two-dimensional blood flow image.

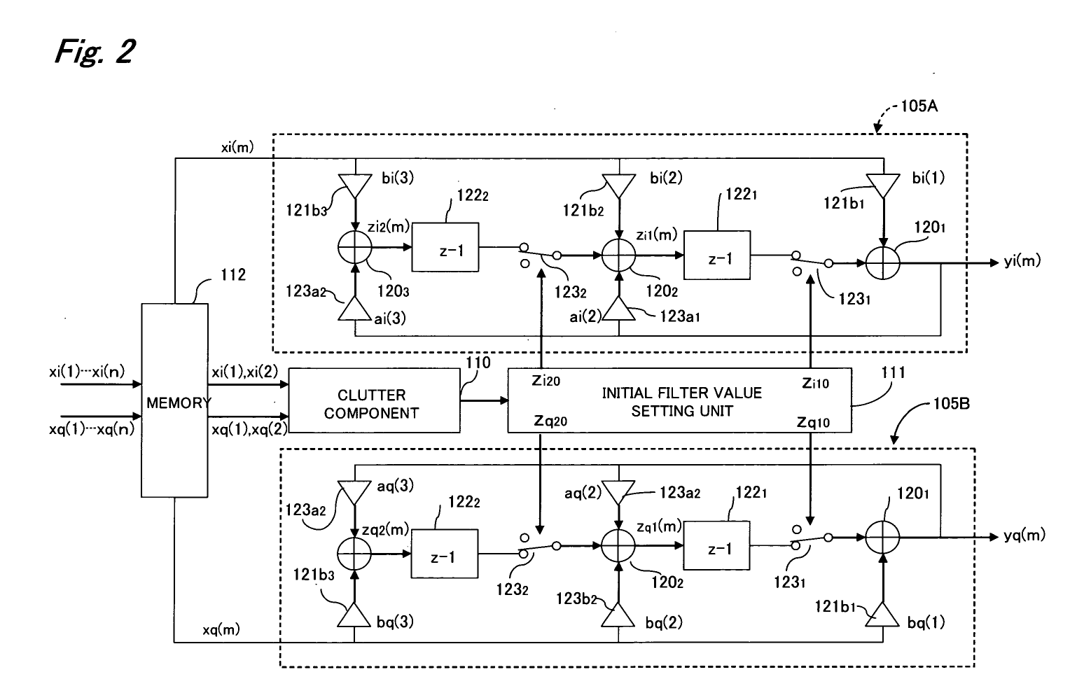

Referring to FIG. 2, structures and operations thereof of the characteristic portions in the blood flow meter 100A will be described in detail below. As shown in the drawing, the blood flow meter 100A has a memory unit 112, which memorizes outputs xᵢ(1)−xᵢ(n) and x_{q}(1)−x_{q}(n) from the phase detector 104. The memory unit 112 transmits the outputs xᵢ(1)−xi(n) and x_{q(1)-x}_{q}(n) into the associated wall filters 105A and 105B and also the first and second outputs xᵢ(1),xᵢ(2), x_{q}(1), and X_{q}(2) into the clutter component estimation unit 110.

Using the first and second outputs xᵢ(1),xᵢ(2), x_{q}(1), and x_{q}(2), the clutter component estimation unit 110 approximates the clutter component contained in the outputs, in the form of sinusoidal wave. In this approximation process, the clutter component estimation unit 110 calculates complex numbers E₁ and E₂ using the following equations (1) and (2):

E₁=xᵢ(1)+j·x_{q}(1) (1)

E₂=xᵢ(2)+j·x_{q}(2) (2)

A phase θc(t) of the sinusoidal wave to be estimated is provided from the following equation (3):

θc(t)=m+n·t (3)

wherein “m” is an initial phase and “n” is a phase velocity.

The initial phase “m” and the phase velocity “n” are provided by the following equations (4) and (5), respectively:

m=angle (E₁) (4)

n=[angle (E₂·E₁*)]/(t₂-T₁) (5)

In those equations, angle (E) is a phase angle of complex number E, E* is a conjugate complex number, and T₁ and T₂ are output timings (corresponding to receipt times) of the detected phases.

An amplitude “a” of the sinusoidal wave is provided from the following equation (6):

a=|E₁| (6)

The clutter component estimation unit 110 uses the amplitude “a” and the phase θc(t) calculated from respective equations to define the sinusoidal wave and transmit a signal corresponding to the sinusoidal wave into the initial filter value generating unit 111. The initial filter value generating unit 111 uses the signal from the clutter component estimation unit 110 to generate initial filter value data zᵢ₁₀, zᵢ₂₀, z_{q10}, and z_{q20}. Those initial filter value data is transmitted to the wall filters 105A and 105B, respectively.

Details of the wall filters 105A and 105B are indicated in FIG. 2. In this drawing, 120₁-120₃ denote adders, 121ₐ₂-121ₐ₃ and 121_{b1}-121_{b3} are multipliers, 122₁ and 122₂ are memory elements (registers), 123₁ and 123₂ are switches, and aᵢ(2)-aᵢ(3) , bⱼ(1)-bⱼ(3) , a_{q}(2)-a_{q}(3) , b_{q}(1)-b_{q}(3) are filter coefficients.

The wall filters 105A and 105B so constructed receive signals corresponding to the initial data zᵢ₁₀, zᵢ₂₀, and z_{q10}, z_{q20} generated at the initial filter value generating unit 111 through switches 123₁ and 123₂ of the filter 105A and the switches 123₁ and 123₂ of the filter 105B, respectively. Once received the initial data, the wall filters 105A and 105B use the initial data zᵢ₁₀, zᵢ₂₀, and z_{q10}, z_{q20} to process respective outputs xᵢ(1)−xᵢ(n) and x_{q}(1)−x_{q}(n) to eliminate the clutter components.

In the arrangement shown in FIG. 2, outputs yᵢ(m) and y_{q}(m) from the wall filters 105A and 105B, and input/output zᵢ₁(m), zᵢ₂(m), and z_{q1}(m), z_{q2}(m) of the wall filters 105A and 105B are defined by the following equations (7) to (12):

yᵢ(m)=bᵢ(1)·xᵢ(m)+zᵢ₁(m−1) (7)

y_{q}(m)=b_{q}(1)·x_{q}(m)+z_{q1}(m−1) (8)

zᵢ₁(m)=bᵢ(2)·xᵢ(m)+zᵢ₂(m−1)−aᵢ(2)·yᵢ(m) (9)

z_{q1}(m)=b_{q}(2)·x_{q}(m)+z_{q2}(m−1)−a_{q}(2)·y_{q}(m) (10)

zᵢ₂(m)=bᵢ(3)·xᵢ(m)−aᵢ(3)·yᵢ(m) (11)

z_{q2}(m)=b_{q}(3)·x_{q}(m)−a_{q}(3)·y_{q}(m) (12)

The wall filters 105A and 105B assign the initial data zᵢ₁₀, zᵢ₂₀, z_{q10}, and z_{q20} to zᵢₗ(m−1), zᵢ₂(m−1) , z_{q1}(m−1), and z_{q2}(m−1) in respective equations (7) to (12). Also, the wall filters use such initial data and another data xᵢ(1) and x_{q}(1) from the memory unit 112 to calculate yᵢ(1) and y_{q}(1), respectively. Likewise, using outputs zᵢ₁(m−1), zᵢ(m−1), z_{q1}(m−1), and z_{q2}(m−1), the m-th output from the phase detector is processed.

As described above, the wall filters 105A and 105B use the initial data generated by approximating the clutter component in the form of sinusoidal wave to obtain the outputs yᵢ(1) and y_{q}(1) for the initial output xᵢ(1) and x_{q}(1), respectively. This decreases possible transient response which could otherwise be generated from the wall filters 105A and 105B, which ensures that the blood flow signal is extracted precisely.

Although the clutter component in the output from the phase detector is approximated in the form of the sinusoidal wave by the use of the first and second outputs xᵢ(1), xᵢ(2), x_{q}(1), and x_{q}(2), the sinusoidal wave may be estimated in the same way by the use of the first and the u-th outputs xᵢ(1), xᵢ(u), x_{q}(1), and x_{q}(u), wherein “u” is an integer which is greater than two but equal to or less than “n”, i.e., n≧u>2. In this instance, the phase velocity of the estimated sinusoidal wave is defined by the following equation (13):

n=[angle (Eᵤ·E₁*)]/(Tᵤ−T₁) (13)

### Second Embodiment

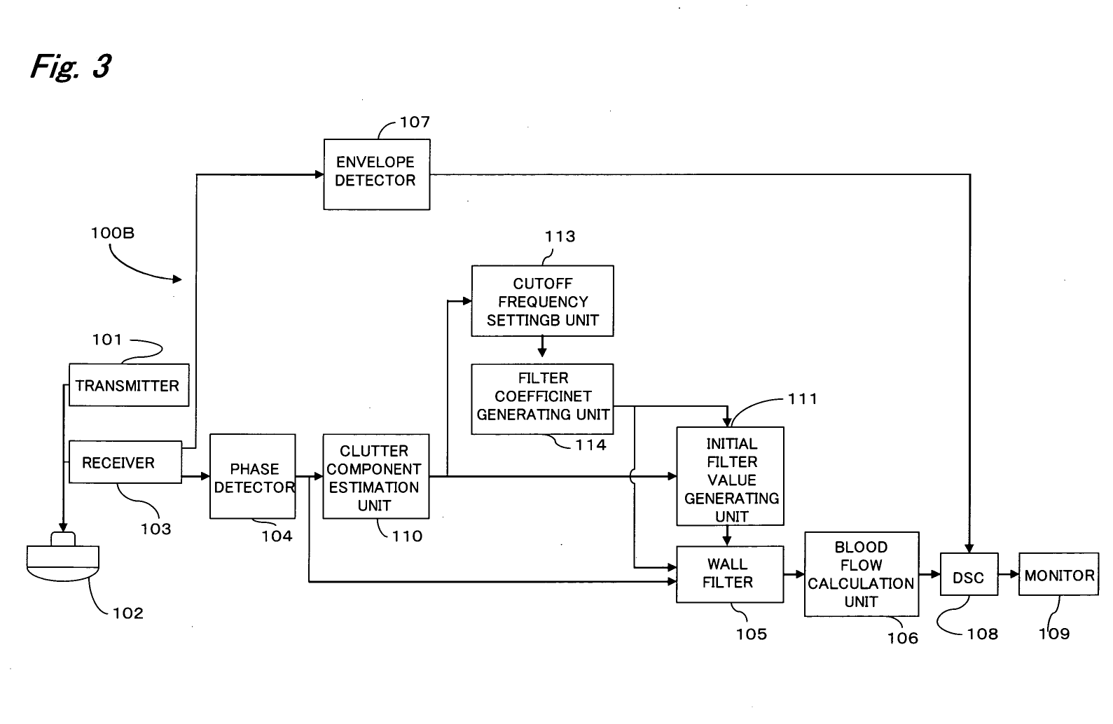

FIG. 3 shows an ultrasonic Doppler blood flow meter, generally indicated at 100B, according to the second embodiment of the present invention. As can be seen when compared with the ultrasonic Doppler blood flow meter 100A of the first embodiment, the illustrated ultrasonic Doppler blood flow meter 100B is characterized in that a cutoff filter frequency setting unit 113 and a filter coefficient generating unit 114 are added thereto.

According to the ultrasonic Doppler blood flow meter 100B, the clutter component estimation unit 110 uses the output from the phase detector to approximate the clutter component by the sinusoidal wave and also estimates the amplitude and the phase velocity of the sinusoidal wave. As described in the first embodiment, estimated information is transmitted into the initial filter value generating unit 111 and the cutoff filter frequency setting unit 113.

Preferably, the ultrasonic Doppler blood flow meter is capable of fully eliminating clutter components and extracting only blood flow data even in the measurements of the portions, such as heart, often delivering rapidly changing clutters and phase velocities. To this end, the cutoff filter frequency setting unit 113 uses the phase velocity and the amplitude to set the lowermost cutoff frequency capable of eliminating the clutter component. The set cutoff frequency is then transmitted into the filter coefficient generating unit 114 in the form of corresponding data. The filter coefficient generating unit 114 generates filter coefficients [aᵢ(2)-aᵢ(2), bᵢ(1)-bᵢ(3), a_{q}(2)-a_{q}(3), and b_{q}(1)-b_{q}(3) in FIG. 2] constituting the filter with the selected cutoff frequency and then delivers those filter coefficients to the wall filter 105. The wall filter 105 uses the filter coefficients to calculate the outputs yᵢ(m) and y_{q}(m).

Although the filter coefficients are fixed in the first embodiment, they may vary with the new blood flow data. Preferably, the initial filer values are updated with the variation of the filter coefficients in the second embodiment. For this purpose, the filer coefficients generated at the filter coefficient generating unit 114 are transmitted into the initial filter value generating unit 111 in the second embodiment. The initial filter value generating unit 111 uses the filter coefficients from the filter coefficient generating unit 114 and the amplitude, the initial phase, and the phase velocity of the clutter approximation estimated at the clutter component estimation unit 110ed to generate the initial filter value for setting the wall filter 105.

The filter coefficient generating unit 114 may calculate the clutter coefficients by using the cutoff frequency according to a predetermined manner or by using a prepared or stored table in which cutoff frequencies are associated with respective filter coefficients.

As described above, according to the ultrasonic Doppler blood flow meter 100B of the second embodiment, only the blood flow is extracted in a stable manner without any clutter to provide highly precise data for the portions, such as heart, delivering rapidly changing clutters and phase velocities.

### Third Embodiment

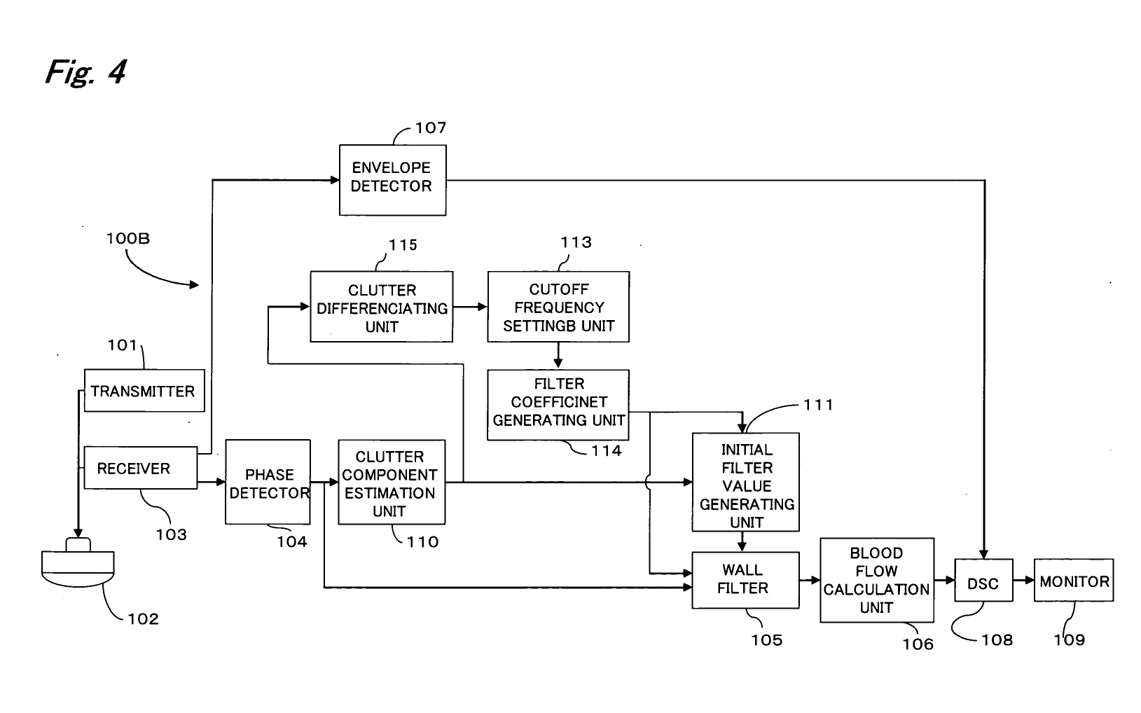

FIG. 4 is a block diagram showing an ultrasonic Doppler blood flow meter, generally indicated at 100C, according to the third embodiment of the present invention. As can be seen when compared with the ultrasonic Doppler blood flow meter 100B according to the second embodiment (see FIG. 3), the ultrasonic Doppler blood flow meter 100C is characterized in that it includes a clutter differentiation unit 115 added thereto.

Typically, according to the ultrasonic diagnostics, the amplitude of the clutter in the echo from the body is several ten times or one-hundred and several ten times greater than that of the blood flow. Contrary to this, the amplitude of the blood flow can be greater than that of the clutter, in the echo signal from portions substantially filled with blood, such as heart and cavities. In this instance, the blood signal with large amplitude can erroneously be identified as clutter and, as a result, removed therefrom according to the method of the second embodiment in which the clutter is identified and then removed.

To prevent this, the clutter differentiation unit 115 in the third embodiment differentiates the clutter from the blood flow data. Specifically, the clutter differentiation unit 115 sets an amplitude threshold. Then, if the amplitude of the approximated sinusoidal wave is greater than the threshold, the unit 115 identifies the estimated sinusoidal wave as clutter and performs subsequent signal processions. If, on the other hand, the amplitude of the estimated sinusoidal wave is equal to or less than the threshold, the unit 115 identifies the estimated sinusoidal wave as the blood flow data and then prohibits processes of the initial filter value generating unit 111, the cutoff frequency setting unit 113 and the filter coefficient generating unit 114 from being performed.

According to the ultrasonic Doppler blood flow meter 100C according to the third embodiment so constructed, even when the amplitude of the blood flow signal from the heart cavity is greater than the clutter signal, the blood flow signal can be extracted reliably to provide precise data.

### Fourth Embodiment

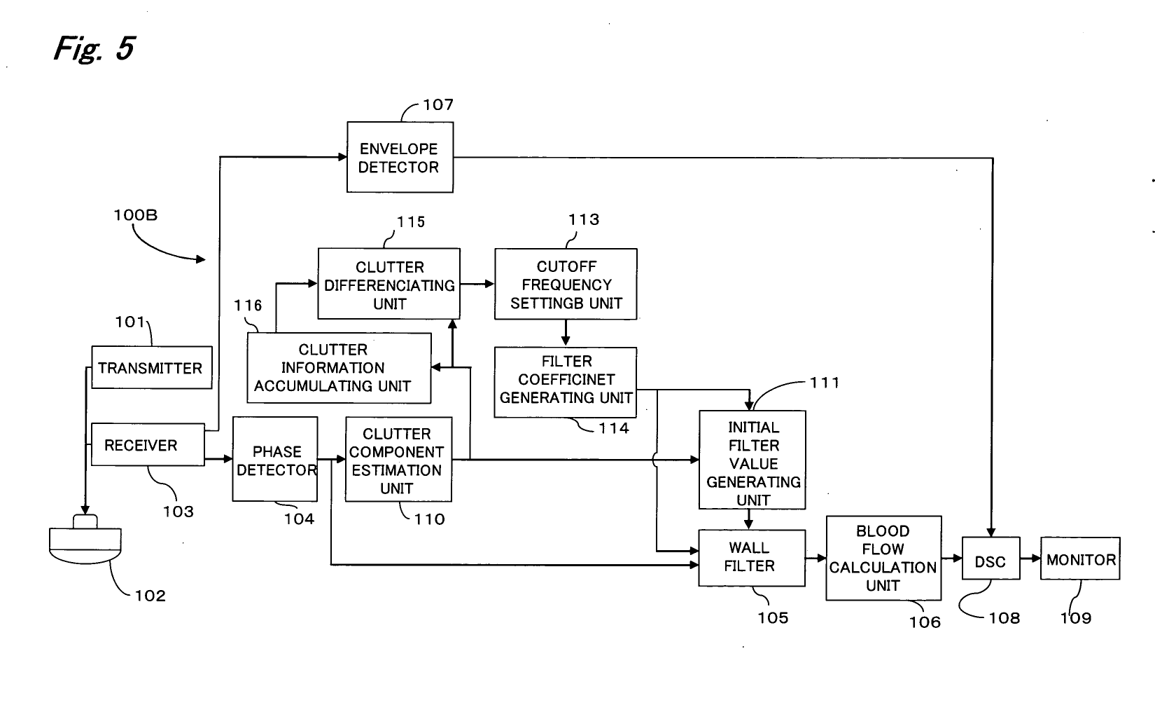

FIG. 5 is a diagram showing an ultrasonic Doppler blood flow meter, generally indicated at 100D, according to the fourth embodiment of the present invention. As can be seen when compared with the ultrasonic Doppler blood flow meter 100C according to the third embodiment (see FIG. 4), the illustrated ultrasonic Doppler blood flow meter 100D is characterized in that it includes a clutter information accumulating unit 115 differentiation unit 116 added thereto.

Specifically, according to the third embodiment, it is determined by the use of the amplitude threshold whether the signal with the greatest amplitude, contained within the echo signal, is the clutter signal or the blood flow data, however, the fourth embodiment is provides with a clutter information accumulating unit 116 as means for setting the threshold.

In the process for scanning incremental portions of the body by the ultrasonic Doppler blood flow meter, it is often occurred that the scanning of one region, adjacent to another region to which the scanning is under way, has already been performed to obtain the clutter and blood flow data or such data has already been obtained by the previously made scanning. Then, in the ultrasonic Doppler blood flow meter 100D of the fourth embodiment, the obtained information is accumulated in the clutter information accumulating unit 116, which is used by the clutter differentiating unit 115 or the clutter information accumulation unit 116 to determine the threshold.

It can not be understood that the clutter signal be always less than the blood flow signal in all regions scanned by the ultrasonic Doppler blood flow meter. Typically, the clutter signal is greater than the blood flow signal, so that the amplitude of the clutter signal is over the range of the blood blow signal. Therefore, the threshold amplitude can be determined by identifying a portion where the clutter signal is greater than the blood flow signal, adjacent to the currently scanning region. Alternatively, it can be predicted how large clutter exists in the currently scanning region by obtaining a space distribution of the clutter amplitude. Also, if the past measurement shows that a large amplitude signal has been detected, for example, by the fact that the scanner moved past over the heart valve, it can be used for the determination of the threshold. As described above, according to the fourth embodiment, the threshold amplitude is determined by the use of accumulated information, allowing reliable blood flow data to be obtained.

1. An ultrasonic Doppler blood flow meter, comprising:
a transmitter for generating electric pulses;
a probe for receiving the electric pulses and transforming the electric pulses, the probe transmitting the transformed electric pulses into a body or subject to be tested and then receiving a signal including an echo signal responsive to the electric pulses from the body;
a phase detector for phase-detecting the echo signal from the signal received by the probe;
a wall filter for eliminating a low frequency clutter component from the echo signal detected by the phase detector;
a blood flow data calculating unit for calculating blood flow data from an output in which the low frequency clutter component has been eliminated;
a digital scan converter for transforming the blood flow data from the blood flow data calculating unit into another form of signal; and
a monitor for using the another form of signal to display blood flow data;
characterized in that
the ultrasonic Doppler blood flow meter further comprises
a clutter component estimation unit for approximating the clutter component by a sinusoidal wave by using at least two signals from the phase detector;
an initial filter value generating unit for generating initial values of the wall filter by using the sinusoidal wave approximated by the clutter component estimation unit;
a cutoff frequency setting unit for setting a cutoff frequency of the wall filter from the estimated clutter component; and
a clutter differentiating unit whether an amplitude of the estimated clutter component indicates the clutter component or the blood flow data from the amplitude information in a region adjacent to another region where it is under scanning or the amplitude information of the clutter component obtained by a previous scanning of the same region.

2-4. (canceled)
